(19) United States
(12) Patent Application Publication
(10) Pub. No.: US 2023/0031813 A1
(43) Pub. Date: Feb. 2, 2023

(54) REVERSIBLE MAGNETS

(71) Applicant: Cochlear Limited, Macquarie University, NSW (AU)

(72) Inventor: Charles Roger Aaron LEIGH, Macquarie University, NSW (AU)

(21) Appl. No.: 17/791,330

(22) PCT Filed: Jan. 20, 2021
(86) PCT No.: PCT/IB2021/050428

§ 371 Date: Jul. 7, 2022

Related U.S. Application Data
(60) Provisional application No. 62/969,802, filed on Feb. 4, 2020.

(51) Int. Cl. A61N 1/378; A61N 1/372; H01F 38/14

(52) U.S. Cl. CPC A61N 1/3787; A61N 1/37217; H01F 38/14; A61N 1/36038

(57) ABSTRACT

A magnetic connection can be formed between a magnet set of a first device and magnet set of a second device. The magnetic strength of the magnetic connection can be modified by changing an orientation of a magnet set of a first device from a first orientation to a second orientation. The magnet set or a case in which the magnet set is disposed can be asymmetric, such that alternating between the first and second orientations results in different magnetic connections.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an example, there is an apparatus comprising a first device having a first device magnet set of one or more magnets configured to provide a magnetic connection to secure the first device relative to a second device. The first device is configured to permit the first device magnet set to be selectively disposable by a user in the first device in first and second opposite orientations to change a strength of the magnetic securing force of the magnetic connection.

In another example, there is a method comprising: establishing a first magnetic connection having a first strength between an implanted magnet set and an external magnet set while the external magnet set is disposed in a first orientation; transitioning the external magnet set from being disposed in the first orientation to being disposed in a second orientation; and establishing a second magnetic connection having a second strength between the implanted magnet and the external magnet set in the second orientation. The first strength is stronger than the second strength. The transitioning includes rotating the external magnet set such that a different face of the external magnet set faces the implanted magnet when the second magnetic connection is established.

In yet another example, there is a system comprising an external device comprising an external magnet set and an implantable device comprising an implantable magnet set. The external magnet set is configured to be selectively disposed in the external device in one of first and second different orientations to change a magnetic strength of a magnetic connection between the external magnet set and the implantable magnet set when the external device is disposed proximate the implantable device.

### BRIEF DESCRIPTION OF THE DRAWINGS

The same number represents the same element or same type of element in all drawings.

FIG. 1 illustrates an example system that includes a first device and a second device having a magnetic connection therebetween.

FIG. 2 illustrates a magnet case disposed in different first and second orientations that change distances between first and second ends of the magnet case and a skin contact surface of the first device.

FIG. 3 illustrates a first example configuration of the magnet case that permits a change in a strength of a magnetic securing force of a magnetic connection with a second magnet set.

FIG. 4 illustrates a second example configuration of the magnet case that permits a change in a strength of a magnetic securing force of a magnetic connection with a second magnet set.

FIG. 5 illustrates a third example configuration of the magnet case that permits a change in a strength of a magnetic securing force of a magnetic connection with a second magnet set.

FIG. 6 illustrates a fourth example configuration of the magnet case that permits a change in a strength of a magnetic securing force of a magnetic connection with a second magnet set.

FIG. 7 illustrates the rotation of the magnet set to transition from a repulsive magnetic connection to an attractive magnetic connection with the second device magnet set.

FIG. 8 illustrates the rotation of the second device magnet set to transition from a repulsive magnetic connection to an attractive magnetic connection with the magnet set.

FIG. 9 illustrates the rotation of the second device magnet set to transition from a repulsive magnetic connection to an attractive magnetic connection with an axially-magnetized magnet set.

FIG. 10 illustrates an example process.

FIG. 11 is a functional block diagram of an implantable stimulator system that can benefit from the technologies described herein.

FIG. 12 illustrates an example cochlear implant system that can benefit from use of the technologies disclosed herein.

FIG. 13 illustrates a retinal prosthesis system that can benefit from use of the technologies disclosed herein.

### DETAILED DESCRIPTION

A magnetic connection can be formed between a magnet set of a first device and magnet set of a second device. Such magnetic connections can be used to, for example, retain the devices in a position relative to each other. As an example use, the first device can be a wearable medical device and the second device can be an implantable medical device. A magnetic connection between the devices can be used to retain the wearable medical device proximate the implantable medical device. A minimum magnetic field strength of the magnetic connection to magnetically retain the wearable medical device in a reliable manner can vary depending on a distance between the devices as a well as the activity level of the wearer. The distance can be affected by a thickness of tissue between the devices, which can vary among recipients of devices. Too great of a magnetic field strength can cause discomfort or damage to the intervening tissue. Desired levels of magnetic field strength can vary not only among individuals, but also a same individual can prefer different magnetic field strengths at different times. The magnetic field strength can be changed by, for example, exchanging one or both of the magnet sets of the devices with a different magnet set. For example, a device can be supplied with different magnet sets for this purpose.

Disclosed examples include techniques to modify a magnetic strength of a magnetic connection between first and second devices by modifying an orientation of a magnet set of a first device. For example, the magnet set or a case in which the magnet set is disposed can be asymmetric, such that alternating between the first and second orientations results in different magnetic connections. This technique can be used to facilitate the changing of magnetic strength without swapping magnet sets.

For example, a first device can include a magnetic element (e.g., a magnet set) for magnetically securing the first device to a magnetic second device. The first device can be configured such that the spatial orientation of the magnetic element relative to the rest of the first device is modifiable. The modification of the spatial orientation can cause a resulting modification of a magnetic securing force between the first device and the second device. The magnetic element can be a set of one or more magnets (e.g. a stack of transversely magnetized magnets such as diametrically magnetized magnets, where the magnets are circular). Where there are two or more magnets, the magnets can have differing magnetic strengths. The set of magnets can be housed in a case.

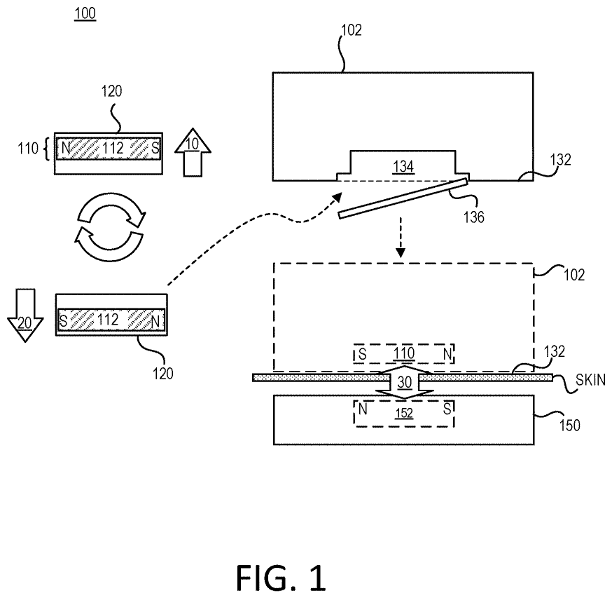

An example implementation of techniques to modify the magnetic strength is described in relation to the example system of FIG. 1.

### Example System

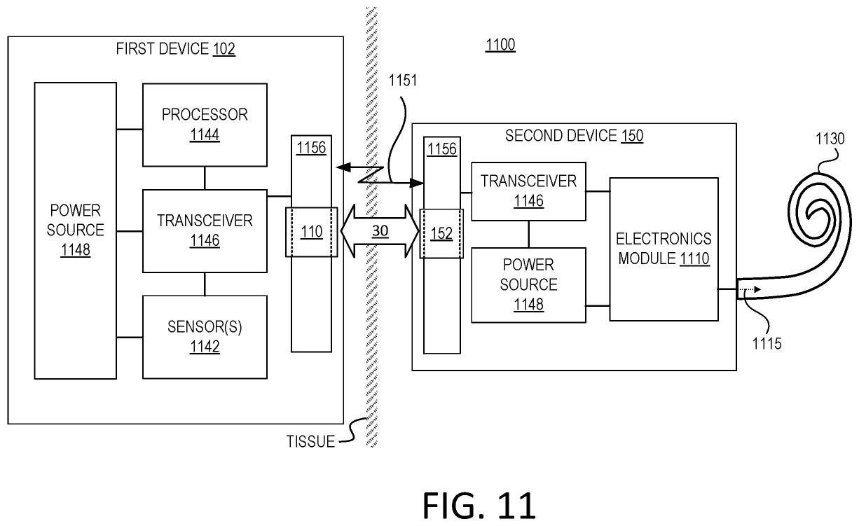

FIG. 1 illustrates an example system 100 that includes a first device 102 and a second device 150 having a magnetic connection 30 therebetween. Any of a variety of devices can benefit from the technology disclosed herein. In the illustrated example, the first device 102 is an external medical device and the second device 150 is an implantable medical device (e.g., an implantable medical stimulator) disposed beneath the recipient's skin. Additional example devices that can benefit from technology disclosed herein are described in more detail in FIGS. 11-13, below.

The first device 102 can include a first device magnet set 110 (which can also be referred to as an external magnet set in examples where the first device is an external device) of one or more magnets. The second device 150 can include a second device magnet set 152 (which can also be referred to as an implanted or implantable magnet set in examples where the second device is an implantable device) of one or more magnets. A magnetic connection 30 can be formed, at least in part, by a magnetic interaction between the first device magnet set 110 and the second device magnet set 152. The magnetic connection 30 can be for any of a variety of purposes, including to secure the first device 102 relative to the second device 150 (e.g., allowing a recipient to wear the first device 102, which can facilitate alignment of coils of the respective devices.

The first device magnet set 110 can be a set of one or more magnets 112 configured to provide a magnetic connection 30 to secure the first device 102 relative to another device. The first device magnet set 110 can be configured to be selectively disposed in the first device 102 in one of first 10 and second 20 different orientations to change a magnetic strength of the magnetic connection 30 between the first device magnet set 110 and the second device magnet set 152 when the first device 102 is disposed proximate the second device 150. The first 10 and second 20 orientations can be opposite each other, such as by being flipped 180-degrees relative to each other.

The first device 102 can be configured to permit the first device magnet set 110 to be selectively disposable by the user in the first device 102 in the first orientation 10 and the second orientation 20. The orientations 10, 20 can be defined with respect to how a portion of the first device magnet set 110 or magnet case 120 is positioned relative to the first device 102 when disposed at the first device 102. This change in orientation can change a strength of the magnetic securing force provided by the magnetic connection 30 when the magnetic connection is established. For example, the first orientation 10 can provide a first magnetic securing force that is less than a second magnetic securing force provided by the second orientation 20.

The one or more magnets 112 can take any of a variety of forms. In the illustrated example, the one or more magnets 112 are transversely magnetized (e.g., diametrically magnetized, where the magnets 112 are disk-shaped). In other examples, the magnets 112 can be axially magnetized. In the illustrated example, the magnet 112 is shown as being rectangular in profile, but the magnet 112 may have other profiles. Further, the magnet 112 and other magnets shown herein are illustrated as being relatively simple (e.g., being uniform in profile) for ease of understanding, but magnets herein can take any of a variety of forms. For instance, a magnet can be formed as a ring or disk of multiple different magnets arranged in various polarities to result in a particular magnetic flux circuit with another magnet set. Magnets herein can, for example, take the form of one or more of the magnets or magnet groups described in US 2019/0239007, which was filed Apr. 5, 2019, is titled “Retention Magnet System for Medical Device”, and is incorporated herein by reference in its entirety for any and all purposes.

In the illustrated example, the first device 102 defines a skin contact surface 132. The skin contact surface 132 can be the portion of the first device 102 that is disposed in contact with the recipient's skin when the first device 102 is in use (e.g., worn by a recipient).

The first device 102 can define a magnet receptacle 134. The magnet receptacle 134 is a portion of the first device 102 configured to receive the first device magnet set 110. For example, the first device 102 can receive the first device magnet set 110 wholly or partially within the magnet receptacle 134. In other examples, the first device magnet set 110 can be disposed outside of (e.g., attached to an outside of) a housing of the first device 102. The magnet receptacle 134 (and thus, the first device magnet set 110 or magnet case 120 therein) can be user-accessible via a magnet cover 136. In an example, the magnet receptacle 134 is defined to receive a magnet case 120 with a circular shape with a diameter arranged parallel to a length of the skin contact surface 132.

FIG. 1 further illustrates that the first device magnet set 110 is removable to permit a user to selectively change the orientation of the first device magnet set 110. For example, the first device magnet set 110 may be in a first orientation 10 within the first device 102. A recipient of the first device 102 can move the magnet cover 136 to expose the magnet case 120 within the magnet receptacle 134. The recipient can remove the magnet case 120, manipulate the magnet case 120, and then return the magnet case 120 to the magnet receptacle 134, such that the first device magnet set 110 is in the second orientation 20. The first device 102 can then be placed in proximity to the second device 150 to form the magnetic connection 30. The resulting magnetic connection 30 can be different from the magnetic connection 30 that existed when the first device magnet set 110 was disposed in the first orientation 10.

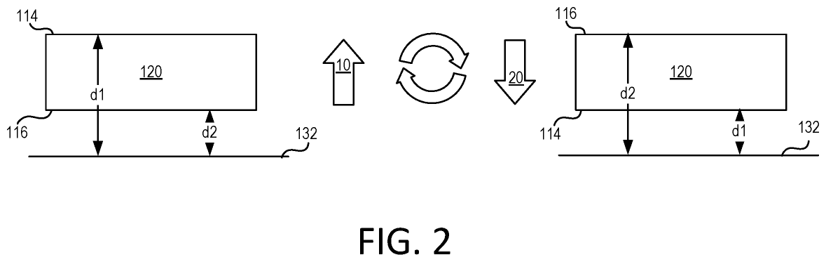

As described above, the differences in the magnetic connection 30 in the first orientation 10 and the second orientation 20 can result from a configuration of the first device magnet set 110. Examples of different configurations of the first device magnet set 110 are shown in FIGS. 2-6.

### Example Magnet Sets

The first device magnet set 110 can be disposed such that the strength of the magnetic connection 30 changes when the first device magnet set 110 placed in different orientations. The magnet case 120 can help keep the magnet sets different relative to each other or otherwise disposed to enable the change in the different orientations.

In the illustrated examples, the magnet case 120 is being shown as being flipped 180 degrees without other modifications. The transitioning from the first orientation 10 to the second orientation 20 can take other forms. In some examples, the magnet case 120 is configured to be removed and replaced such that a particular pole faces a same direction in the first 10 and second 20 orientations (e.g., to cooperate with a second device magnet set 152 to result in a desired attractive or repulsive magnetic connection 30). For example, the magnet case 120 can have a recess configured to cooperate with a tab extending into the magnet receptacle 134 to orient the magnet case 120 such that a particular pole is in a particular orientation.

In examples, the one or more components of the first device magnet set 110 or within the magnet case 120 can be modifiable. For example, the relative size of different volumes can be changed. The relative distance or angle between components can be changed. Examples of arrangements for modifying such components are described in US 2015/0382114, which was filed on Jun. 25, 2014, which is titled “System for Adjusting Magnetic Retention Force in Auditory Prostheses”, and which is hereby incorporated herein by reference for any and all purposes.

FIG. 2 illustrates a magnet case 120 disposed in different first 10 and second 20 orientations, which results in a change in distances between a first end 114 and a second end 116 of the magnet case 120 and a skin contact surface 132 of the first device 102. As illustrated, there is a first distance d1 between the first end 114 and the skin contact surface 132 and a second distance d2 between the second end 116 and the skin contact surface 132. When the magnet case 120 is disposed in the first orientation 10, the first distance d1 is greater than the second distance d2 and the second end 116 faces the skin contact surface 132. When the magnet case 120 is disposed in the second orientation 20, the second distance d2 is greater than the first distance d1 and the first end 114 faces the skin contact surface 132. In the illustrated example, distance d1 in the first orientation 10 is equal to d2 in the second orientation 20, and distance d2 in the first orientation 10 is equal to d1 in the second orientation 20. In other examples, the distances need not be equal. These differences can define the orientations 10, 20. For example, the first orientation 10 can be defined as a particular distance or a particular end of the magnet case 120 or first device magnet set 110 facing a particular surface. And the second orientation 20 can be defined as a particular different distance or different end facing the particular surface. These differences between the first 10 and second 20 orientations cause a change in the strength of the magnetic connection 30 based on a configuration of the magnet case 120 or the first device magnet set 110.

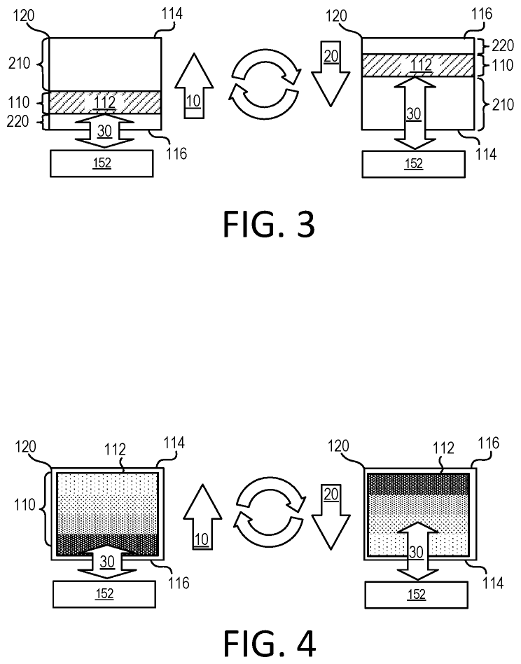

The differences in distances d1 and d2 can also relate to distances between the magnets disposed in the magnet case 120 and the skin-contact surface 132. For example, the magnet case 120 can define a logical or actual first half and a second half, and the change in orientation can have a corresponding change in which half is closer to the skin-contact surface 132 (and thus the second device magnet set 152). Differences in magnetic properties of the first and second halves can change a resulting magnetic connection 30 provided by the magnet case 120. For example, where the first half is more magnetic than the second half, the magnetic connection 30 can have its strength modified depending on which half is closer to the skin contact surface 132. Example configurations are described in more detail below in relation to FIGS. 3-6.

FIG. 3 illustrates a first example configuration of the magnet case 120 that permits a change in a strength of a magnetic securing force of a magnetic connection 30 with a second device magnet set 152 in different first and second orientations 10, 20. In the first example configuration, the magnet case 120 defines a first volume 210 and a second volume 220 between which the first device magnet set 110 having a single magnet 112 is disposed. Here, the first volume 210 is larger than the second volume 220. As a result, when the magnet case 120 is disposed such that the second volume 220 is between the magnet 112 and the second magnet set 152 (as in the first orientation 20), then the distance between the magnet 112 and the second magnet set 152 is smaller than when the first volume 210 is between the magnet 112 and the second magnet set 152 (as in the second orientation 20). The magnet case 120 can be transitioned from the first orientation 10 to the second orientation by rotating the magnet case 120 to change which volume will be disposed between the magnet 112 and second magnet set 152. The first volume 210 and the second volume 220 can lack a magnetic material. The volumes 210, 220 can be formed from a non-magnetic material, such as plastic or air. In some examples, the volumes 210, 220 can include one or more components configured to attenuate or modify a magnetic flux produced by the first device magnet set 110 so as to modify the magnetic strength provided in the first and second orientation 10, 20.

FIG. 4 illustrates a second example configuration of the magnet case 120 that permits a change in a strength of a magnetic securing force of a magnetic connection 30 with a second magnet set 152 in different first and second orientations 10, 20. In the second example configuration, the properties of the one or more magnets 112 of the first device magnet set 110 cause the change in the strength. Here, the strength of the first device magnet set 110 varies across the first device magnet set 110. As illustrated, the first device magnet set 110 include a single magnet having an axial change in magnetic strength. In other examples, the first device magnet set 110 can include multiple magnets 112 of varying strength. As a result of the varying strength, when the magnet case 120 is disposed in the first orientation 10, the stronger portion of the first device magnet set 110 is closer to the second magnet set 152 than the stronger portion is in the second orientation 20, thus changing a strength of the magnetic connection 30. The magnet case 120 can be transitioned from the first orientation 10 to the second orientation 30 by rotating the magnet case 120 to change which portion of the of the first device magnet set 110 is closer to the second magnet set 152.

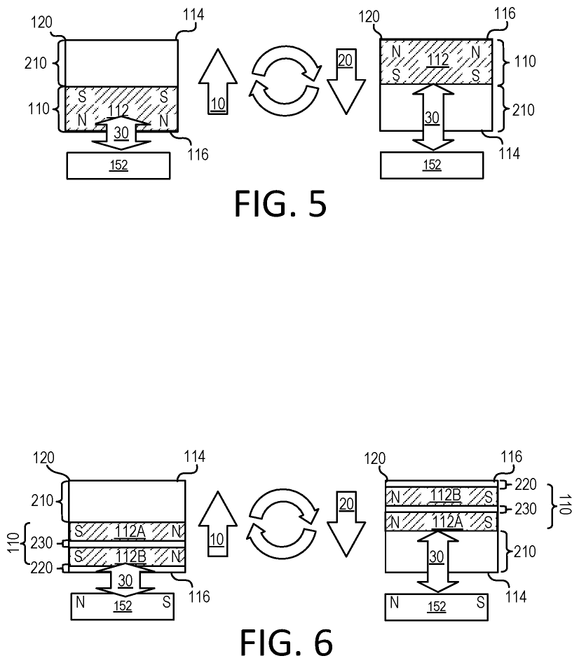

FIG. 5 illustrates a third example configuration of the magnet case 120 that permits a change in a strength of a magnetic securing force of a magnetic connection 30 with a second magnet set 152 in different first and second orientations 10, 20. In the third example configuration, the magnet case 120 defines a first volume 210 between the first device magnet set 110 and the first end 114 of the magnet case 120. The first device magnet set 110 can be disposed proximate the second end 116 of the magnet case 120. The first device magnet set 110 can take up a substantial remainder of the volume within the magnet case 120 that is not taken up by the first volume 210. As a result, when the magnet case 120 is disposed such that the first volume 210 is disposed away from the second magnet set 152 (as in the first orientation 10), then the distance between the magnet 112 and the second magnet set 152 is smaller than when the first volume 210 is between the magnet 112 and the second magnet set 152 (as in the second orientation 20). The magnet case 120 can be transitioned from the first orientation 10 to the second orientation by rotating the magnet case 120 to change whether the first volume 210 is disposed between the magnet 112 and second magnet set 152. In the illustrated example, the first device magnet set 110 is axially magnetized. In other examples, the first device magnet set 110 can be transversely magnetized. The first device magnet set 110 can use magnets with poles disposed in other ways such as South above North on one side and North above South on the other. Examples of multi-pole magnet configurations are described in U.S. provisional patent application No. 62/907,044, which is hereby incorporated by reference in its entirety for any and all purposes.

FIG. 6 illustrates a fourth example configuration of the magnet case 120 that permits a change in a strength of a magnetic securing force of a magnetic connection 30 with a second magnet set 152 in different first and second orientations 10, 20. In the fourth example configuration, the magnet case 120 defines a first volume 210 and a second volume 220 between which the first device magnet set 110 is disposed. The first device magnet set 110 includes a first magnet 112A and a second magnet 112B. The first magnet 112A and the second magnet 112B can be disposed such that their respective north poles are proximate each other. Where like poles of the first magnet 112A and the second magnet 112B are proximate each other, magnetic force can urge the magnets away from this position. The magnet case 120 can be constructed to hold the magnets in the position with their respective north poles proximate each other. The first magnet 112A and the second magnet 112B can have a same or different magnetic strength. For example, the first magnet 112A can have a greater magnetic strength than the second magnet 112B. The magnet case 120 further defines a third volume 230 that is disposed between the first magnet 112A and the second magnet 112B. As with the first example configuration, in this illustrated examples, the first volume 210 is larger than the second volume 220. As a result, when the magnet case 120 is disposed such that the second volume 220 is between the magnet 112 and the second magnet set 152 (as in the first orientation 10), then the distance between the magnet 112 and the second magnet set 152 is smaller than when the first volume 210 is between the magnet 112 and the second magnet set 152 (as in the second orientation 20). The magnet case 120 can be transitioned from the first orientation 10 to the second orientation by rotating the magnet case 120 to change which volume will be disposed between the magnet 112 and second magnet set 152, thus modifying the strength of the magnetic connection 30. In other examples, a relative difference in strength of the magnets 112A, 112B can partially or wholly contribute to the change in strength of the magnetic connection 30.

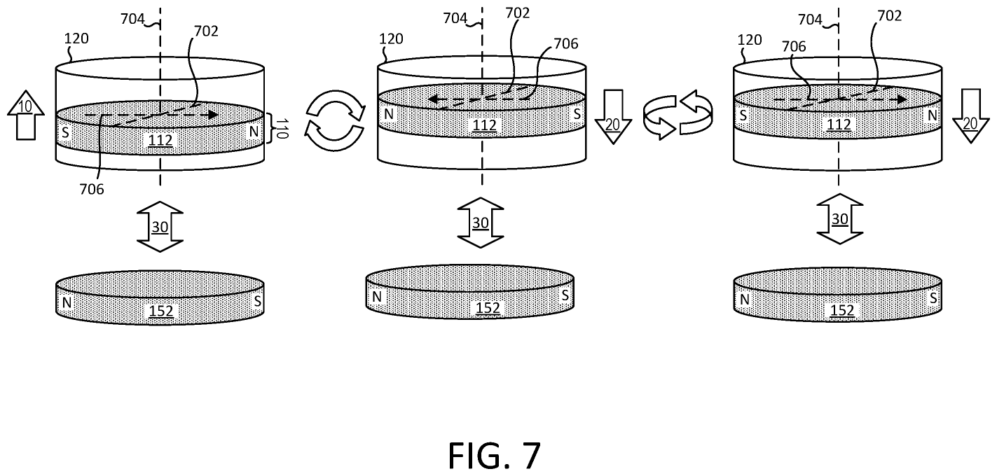

FIG. 6 further illustrates that, in the first orientation, the south poles of the first device magnet set 110 are disposed proximate one or more north poles of the second device magnet set 152. As a result of the transitioning from the first orientation 10 to the second orientation 20, the magnets 112 of the first device magnet set 110 are flipped such that the south poles of the first device magnet set 110 are proximate the south poles of the second device magnet set 152. This results in the formerly attracting force caused by the magnetic connection 30 in the first orientation 10 to become repulsive force in orientation 20. In certain applications, it can be desirable for there to be attraction in both the first and second orientations 10, 20 or repulsion in both the first and second orientations 10, 20, rather than the mixing of the two as described above. Techniques for moving the poles to adjust for the change from the first orientation 10 to the second orientation 20 are described below in relation to FIGS. 7-9.

### Example Pole Movement

As described above in relation to FIG. 6, the transitioning from a first orientation 10 to a second orientation 20, without further modification, can result in the flipping of poles of the first device magnet set 110. This flipping can result in changing the magnetic connection 30 to be repulsion rather than attraction (or vice versa). To correct for this, one or more of the magnets 112 of the first device magnet set 110 or the second device magnet set 152 can be rotatable. The rotation can allow for self-correction of a repulsive magnetic connection 30 into an attractive magnetic connection 30. For instance, the first device magnet set 110 or the second device magnet set 152 can be freely-rotatable (e.g., sufficiently low friction when disposed in their respective devices) such that strength of the magnetic connection causes the rotation. The magnet sets 110, 152 can be rotatable by a user.

FIG. 7 illustrates the rotation of the first device magnet set 110 to transition from a repulsive magnetic connection 30 to an attractive magnetic connection 30 with the second device magnet set 152. The illustrated magnet case 120 is disk-shaped with a first device magnet set 110 disposed therein. The one or more magnets 112 of the first device magnet set 110 define a diameter 702 and an axis 704 perpendicular to the diameter 702. The one or more magnets 112 of the first device magnet set 110 include a magnetization direction 706. In the illustrated example, the magnet 112 of the first device magnet set 110 is transversely magnetized. More particularly, the magnet 112 has a circular shape defining the diameter 702, and the magnet is diametrically magnetized. The magnet 112 has a magnetization direction 706 parallel to a plane formed by the diameter 702 of the first device magnet set 110. The first device magnet set 110 (e.g., the magnets 112 thereof) can be rotatable about the axis 704. For example, the first device magnet set 110 can be configured to be rotatable within the magnet case 120. In addition or instead, the magnet case 120 can be configured to be rotatable about the axis 704 (e.g., within the magnet receptacle 134), thereby rotating the first device magnet set 110 disposed within the magnet case 120. In yet another example, the entire device in which the magnet case 120 is disposed (e.g., the first device 102) rotates (e.g., under a recipient's manual force or under the force of the magnetic connection 30) until desired pole alignment is achieved for the magnets disposed in the device and the implanted magnet set 152 is achieved. The components can be configured to be rotatable by, for example, having play within a surrounding area. In addition or instead, the components can be configured with sufficiently low friction (e.g., having a low-friction coating, being made from a low friction material, or being used with one or more bearings to facilitate rotation) to permit a desired force to rotate the component.

As illustrated, the transitioning from the first orientation 10 to the second orientation 20 can result in the one or more north poles of the first device magnet set 110 can being disposed proximate the one or more north poles of the second device magnet set 152, which can result in a repulsive magnetic connection 30. With the first device magnet set 110 being configured to rotate, the force of the magnetic connection 30 can cause the first device magnet set 110 to rotate, resulting in the one or more north poles of the first device magnet set 110 being proximate one or more south poles of the second device magnet set 152.

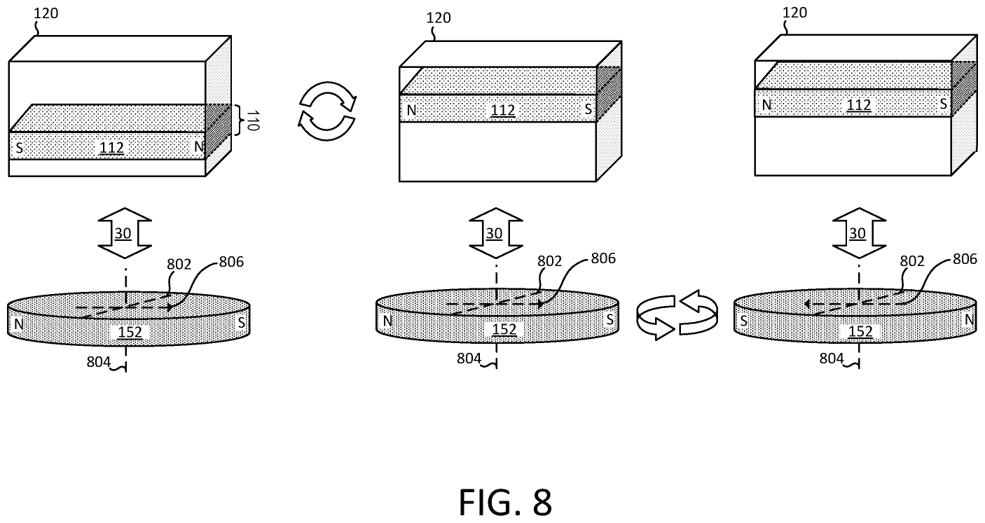

FIG. 8 illustrates the rotation of the second device magnet set 152 to transition from a repulsive magnetic connection 30 to an attractive magnetic connection 30 with the first device magnet set 110. The illustrated magnet case 120 is rectangular with a first device magnet set 110 disposed therein. The magnet case 120 can be configured to (e.g., by its construction) resist rotation of the first device magnet set 110. In another example, the first device magnet set 110 defines a diameter and an axis perpendicular to the diameter, and the first device magnet set 110 is configured to resist rotation about the diameter. The second device magnet set 152 is disk-shaped and defines a diameter 802 and an axis 804 perpendicular to the diameter 802. The second device magnet set 152 includes a magnetization direction 806. The magnetization direction 806 indicates that the second device magnet set 152 is transversely magnetized. In particular, the second device magnet set 152 is diametrically magnetized. The second device magnet set 152 is magnetized in a direction parallel to a plane formed by the diameter 802 of the second device magnet set 152. The second device magnet set 152 (e.g., the magnets thereof) can be rotatable about the axis 804. For example, the second device magnet set 152 can be configured to be rotatable within a magnet case. The components can be configured to be rotatable by, for example, having play within a surrounding area. In addition or instead, the components can be configured with low friction (e.g., having a low-friction coating, being made from a low friction material, or being used with one or more bearings to facilitate rotation).

As illustrated, the transitioning from the first orientation 10 to the second orientation 20 can result in the one or more north poles of the first device magnet set 110 being disposed proximate the one or more north poles of the second device magnet set 152, which can result in a repulsive magnetic connection 30. With the second device magnet set 152 being configured to rotate, the force of the magnetic connection 30 can cause the second device magnet set 152 to rotate, resulting in the one or more north poles of the second device magnet set 152 being proximate one or more south poles of the first device magnet set 110.

The examples provided in FIGS. 7 and 8 show the first device magnet set 110 and the second device magnet set 152 being transversely magnetized. Pole movement can also be used to correct the attraction and repulsion provided by the magnetic connection 30 where the magnet set is axially magnetized. In addition, while FIGS. 7 and 8 show only one of the two magnet sets 110, 152 moving (e.g., rotating), in examples, both the magnet sets can be configured to rotate.

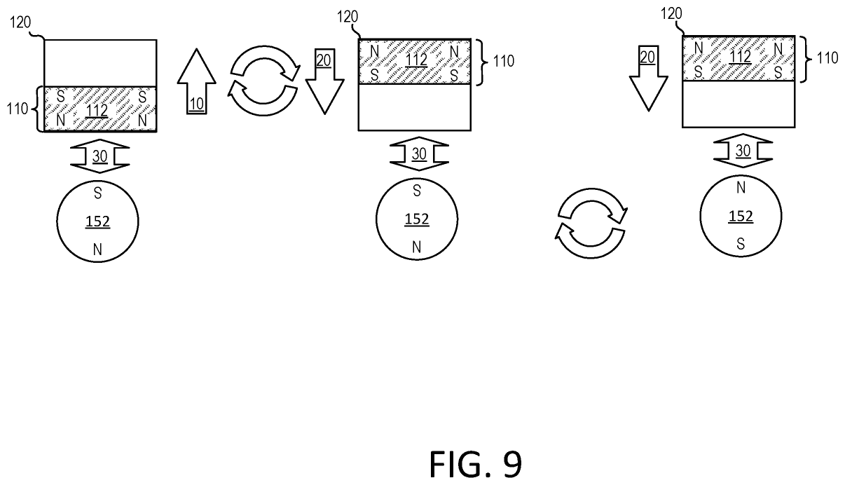

FIG. 9 illustrates the rotation of the second device magnet set 152 to transition from a repulsive magnetic connection 30 to an attractive magnetic connection 30 with an axially-magnetized first device magnet set 110. The illustrated magnet case 120 has a rectangular profile with a first device magnet set 110 disposed therein. The first device magnet set 110 is axially magnetized. The second device magnet set 152 is disk-shaped and is diametrically magnetized. Further, the second device magnet set 152 is configured to be rotatable such that either pole can face the first device magnet set 110. The components can be configured to be rotatable by, for example, having play within a surrounding area. In addition or instead, the components be configured with low friction (e.g., having a low-friction coating, being made from a low friction material, or being used with one or more bearings to facilitate rotation).

As illustrated, the transitioning from the first orientation 10 to the second orientation 20 can result in the one or more south poles of the first device magnet set 110 being disposed proximate the one or more north poles of the second device magnet set 152, which can result in a repulsive magnetic connection 30. With the second device magnet set 152 being configured to rotate, the force of the magnetic connection 30 can cause the second device magnet set 152 to rotate, resulting in the one or more north poles of the first device magnet set 110 being proximate one or more south poles of the second device magnet set 152, thereby changing the magnetic connection 30 being attractive rather than repulsive. In the illustrated example, the axis of rotation of the second device magnet set 152 can be substantially perpendicular to the magnetization direction of the first device magnet set 110.

### Example Process

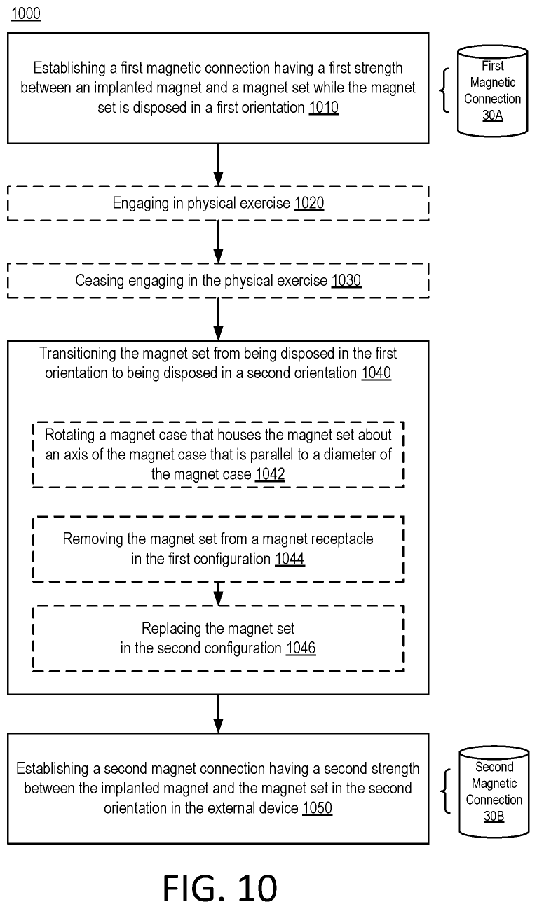

FIG. 10 illustrates an example process 1000. The process begins with operation 1010.

Operation 1010 includes establishing 1010 a first magnetic connection 30A having a first strength between an implanted magnet set 152 (e.g., a second device magnet set 152 as described above) and an external magnet set 110 while the external magnet set 110 (e.g., a first device magnet set 110 as described above) is disposed in a first orientation 10. In some examples, establishing the first magnetic connection 30A includes wearing an external device in which the external magnet set 110 is disposed. In some examples, establishing 1010 the first magnetic connection 30A includes establishing an inductive power and data connection between an external device in which the external magnet set 110 is disposed and an implanted device 150 associated with the implanted magnet set 152. In some examples, establishing 1010 the first magnetic connection 30A includes bringing the external magnet set 110 in proximity to the implanted device 150.

In some examples, following operation 1010, the flow of the process 1000 moves to operation 1040. In other examples, the flow of the process 1000 moves to operation 1020.

Operation 1020 includes engaging in physical exercise. For example, the recipient of the first device 102 can engage in physical exercise while wearing the first device 102. During the physical exercise, the external magnet set 110 can have the first magnetic connection 30A with the second device magnet set 152. The first magnetic connection 30A can be tuned to be sufficiently strong to help retain the first device 102 during physical exercise. Following operation 1020, the flow of the process moves to operation 1030.

Operation 1030 includes ceasing engaging in physical exercise. For example, the recipient of the first device 102 stops the physical exercise. In response to ceasing engaging in the physical exercise, the flow of the process 1000 can move to operation 1040.

Operation 1040 includes transitioning the external magnet set 110 from being disposed in the first orientation 10 to being disposed in a second orientation 20. In some examples, operation 1040 includes operation 1042. In some examples, operation 1040 includes operations 1044 and 1046.

Operation 1042 includes rotating the external magnet set 110 such that a different face of the magnet case 120 faces the implanted magnet set 152 when the second magnetic connection 30B is established. In some examples, rotating 1042 the magnet case 120 includes rotating the magnet case 120 by 180 degrees. This rotation can be performed by, for example, the recipient of the first device 102. In other examples, the device 102 itself causes the rotation (e.g., with an actuator).

Operation 1044 includes removing the external magnet set 110 from a magnet receptacle 134 in the first orientation 10. Depending on the relationship between the external magnet set 110 and the first device 102, this can occur in any of a variety of ways. In an example, the operation 1044 includes removing a magnet case 120 in which the external magnet set 110 is disposed, thereby removing the external magnet set 110. In some examples, this operation 1044 can include removing a component keeping the external magnet set 110 in the magnet receptacle 134. For example, the operation can include removing a cover 136 to access the external magnet set 110. In another example, following operation 1044, the flow of the process 1000 can move to operation 1046.

Operation 1046 includes replacing 1046 the external magnet set 110 in the magnet receptacle 134 in the second orientation 20. In some examples, this operation 1046 further includes securing the external magnet set 110 (or a magnet case 120 in which the external magnet set 110 is disposed) in the magnet receptacle 134. For example, after the external magnet set 110 is placed in the receptacle 134, a cover 136 can be applied to secure the external magnet set 110 therein. In another example, there can be a threaded connection between the magnet case 120 and the magnet receptacle 134 such that the magnet case 120 can be rotatably locked or screwed into the magnet receptacle 134.

Following operation 1040, the flow of the process 1000 can move to operation 1050.

Operation 1050 includes establishing a second magnetic connection 30B having a second strength between the implanted magnet 152 and the external magnet set 110 in the second orientation 20. For example, this operation 1050 can be similar to or the same as described in operation 1010 except that the external magnet set 110 is in the second orientation 20. The first strength can be stronger than the second strength. In another example, the first strength can be weaker than the second strength.

### Example Devices

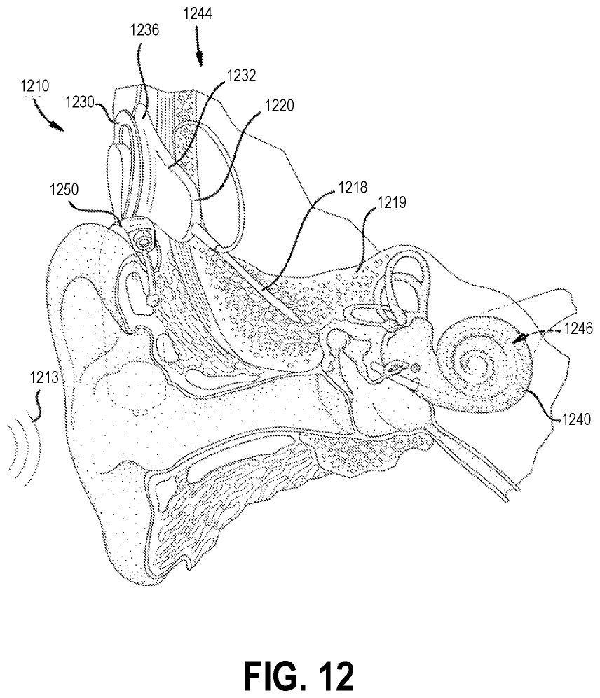

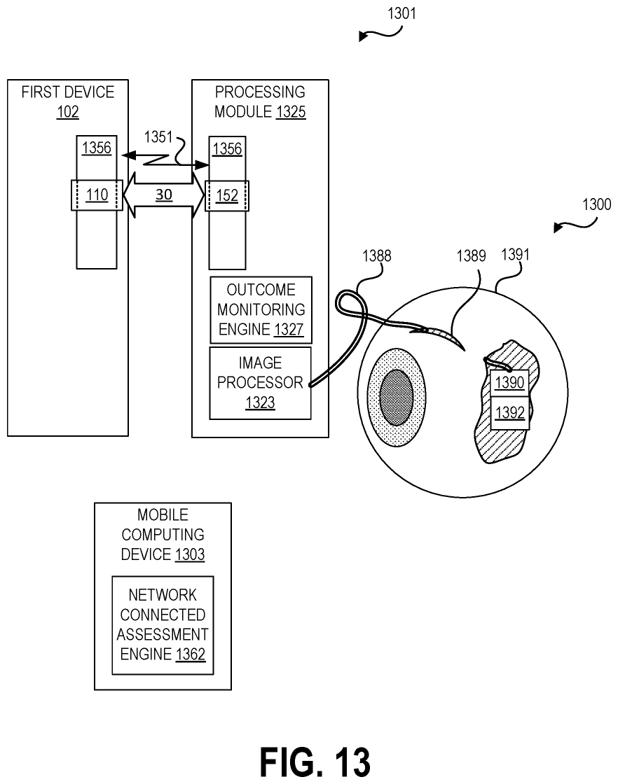

As previously described, the technology disclosed herein can be applied in any of a variety of circumstances and with a variety of different devices. Example devices that can benefit from technology disclosed herein are described in more detail in FIGS. 11-13, below. For example, the system 100 can be or be part of a medical stimulation system 1100, such as is shown in FIG. 11. In an example, the first device 102 can be a wearable medical device and the second device 150 can be an implantable medical stimulator of an auditory prosthesis, such as a cochlear implant as described in FIG. 12. As another example, the first device 102 and the second device 150 can cooperate to act as a retinal prosthesis, such as is described in FIG. 13. The technology can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. These different systems and devices can benefit from the technology described herein.

### Example Implantable Stimulation System

FIG. 11 is a functional block diagram of an implantable stimulator system 1100 that can benefit from the technologies described herein. The implantable stimulator system 1100 includes the first device 102 acting as an external processor device and the second device 150 acting as an implanted stimulator device. In examples, the second device 150 is an implantable stimulator device configured to be implanted beneath a recipient's tissue (e.g., skin). In examples, the second device 150 includes a biocompatible housing. The first device 102 is a device configured to couple with (e.g., wirelessly) the second device 150 to provide additional functionality.

In the illustrated example, the first device 102 includes one or more sensors 1142, a processor 1144, a transceiver unit 1146, and a power source 1148. The one or more sensors 1142 can be units configured to produce data based on sensed activities. In an example where the stimulation system 1100 is an auditory prosthesis system, the one or more sensors 1142 can include sound input sensors, such as a microphone, an electrical input for an FM hearing system, and/or another component for receiving sound input. Where the stimulation system 1100 is a visual prosthesis system, the one or more sensors 1142 can include one or more cameras or other visual sensors. Where the stimulation system 1100 is a cardiac stimulator, the one or more sensors 1142 can include cardiac monitors. The processor 1144 can be a component (e.g., a central processing unit) configured to control stimulation provided by the second device 150. The stimulation can be controlled based on data from the sensor 1142, a stimulation schedule, or other data. Where the stimulation system 1100 is an auditory prosthesis, the processor 1144 can be configured to convert sound signals received from the sensor(s) 1142 (e.g., acting as a sound input unit) into external device signal 1151. The transceiver 1146 is configured to send power signals 1151, data signals 1151, combinations thereof (e.g., by interleaving the signals), or other signals. The transceiver 1146 can also be configured to receive power or data. Stimulation signals can be generated by the processor 1144 and transmitted, using the transceiver 1146, to the second device 150 for use in providing stimulation.

In the illustrated example, the second device 150 includes an electronics module 1110, a stimulator assembly 1130, a transceiver 1140, a power source 1148, and a coil 1156. The second device 150 further includes a hermetically sealed, biocompatible housing enclosing one or more of the components.

The electronics module 1110 can include one or more other components to provide auditory prosthesis functionality. In many examples, the electronics module 1110 includes one or more components for receiving a signal and converting the signal into the stimulation signal 1115. The electronics module 1110 can further include a stimulator unit. The electronics module 1110 can generate or control delivery of the stimulation signals 1115 to the stimulator assembly 1130. In examples, the electronics module 1110 includes one or more processors (e.g., central processing units) coupled to memory components (e.g., flash memory) storing instructions that when executed cause performance of an operation described herein. In examples, the electronics module 1110 generates and monitors parameters associated with generating and delivering the stimulus (e.g., output voltage, output current, or line impedance). In examples, the electronics module 1110 generates a telemetry signal (e.g., a data signal) that includes telemetry data. The electronics module 1110 can send the telemetry signal to the first device 102 or store the telemetry signal in memory for later use or retrieval.

The stimulator assembly 1130 can be a component configured to provide stimulation to target tissue. In the illustrated example, the stimulator assembly 1130 is an electrode assembly that includes an array of electrode contacts disposed on a lead. The lead can be inserted into the recipient's cochlea. The stimulator assembly 1130 can be configured to deliver stimulation signals 1115 (e.g., electrical stimulation signals) generated by the electronics module 1110 to the cochlea to cause a hearing percept in the recipient. In other examples, the stimulator assembly 1130 is a vibratory actuator disposed inside or outside of a housing of the second device 150 and configured to generate vibrations. The vibratory actuator receives the stimulation signals 1115 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 1146 can be components configured to transcutaneously receive and/or transmit a signal 1151 (e.g., a power signal and/or a data signal). The transceiver 1146 can be a collection of one or more components that form part of a transcutaneous energy or data transfer system to transfer the signal 1151 between the first device 102 and the second device 150. Various types of signal transfer, such as electromagnetic, capacitive, and inductive transfer, can be used to usably receive or transmit the signal 1151. The transceiver 1146 can include or be electrically connected to the coil 1156.

The coils 1156 can be components configured to receive or transmit a signal 1151, typically via an inductive arrangement formed by multiple turns of wire. In examples, in addition to or instead of a coil, other arrangements can be used, such as an antenna or capacitive plates. The magnet sets 110 and 152 can be used to align respective coils 1156 of the first device 102 and the second device 150. For example, the coil 1156 of the second device 150 can be disposed in relation to (e.g., in a coaxial relationship) with the second device magnet set 152 to facilitate orienting the coil 1156 in relation to the coil 1156 of the first device 102 via the magnetic connection 30. The coil 1156 of the first device 102 can be disposed in relation to (e.g., in a coaxial relationship) with the external magnet set 110.

The power sources 1148 can be components configured to provide operational power to other components. The power sources 1148 can be or include one or more rechargeable batteries. Power for the batteries can be received from a source and stored in the battery. The power can then be distributed to the other components of the second device 150 as needed for operation.

As should be appreciated, while particular components are described in conjunction with FIG. 11, technology disclosed herein can be applied in any of a variety of circumstances. The above discussion is not meant to suggest that the disclosed techniques are only suitable for implementation within systems akin to that illustrated in and described with respect to FIG. 11. In general, additional configurations can be used to practice the methods and systems herein and/or some aspects described can be excluded without departing from the methods and systems disclosed herein.

### Example Implantable Stimulation System—Cochlear Implant System

FIG. 12 illustrates an example cochlear implant system 1210 that can benefit from use of the technologies disclosed herein. The cochlear implant system 1210 includes an implantable component 1244 (e.g., the second device 150) typically having an internal receiver/transceiver unit 1232, a stimulator unit 1220, and an elongate lead 1218. The internal receiver/transceiver unit 1232 permits the cochlear implant system 1210 to receive signals from and/or transmit signals to an external device 1250 (e.g., the first device 102). The external device 1250 can be a button sound processor worn on the head that includes a receiver/transceiver coil 1230 and sound processing components. Alternatively, the external device 1250 can be just a transmitter/transceiver coil in communication with a behind-the-ear device that includes the sound processing components and microphone.

The implantable component 1244 includes an internal coil 1236, and preferably, a magnet (not shown) fixed relative to the internal coil 1236. The magnet can be embedded in a pliable silicone or other biocompatible encapsulant, along with the internal coil 1236. Signals sent generally correspond to external sound 1213. The internal receiver/transceiver unit 1232 and the stimulator unit 1220 are hermetically sealed within a biocompatible housing, sometimes collectively referred to as a stimulator/receiver unit. Included magnets can facilitate the operational alignment of an external coil 1230 and the internal coil 1236 (e.g., via a magnetic connection 30 generated using techniques described herein), enabling the internal coil 1236 to receive power and stimulation data from the external coil 1230. The external coil 1230 is contained within an external portion. The elongate lead 1218 has a proximal end connected to the stimulator unit 1220, and a distal end 1246 implanted in a cochlea 1240 of the recipient. The elongate lead 1218 extends from stimulator unit 1220 to the cochlea 1240 through a mastoid bone 1219 of the recipient. The elongate lead 1218 is used to provide electrical stimulation to the cochlea 1240 based on the stimulation data. The stimulation data can be created based on the external sound 1213 using the sound processing components and based on sensory prosthesis settings.

In certain examples, the external coil 1230 transmits electrical signals (e.g., power and stimulation data) to the internal coil 1236 via a radio frequency (RF) link. The internal coil 1236 is typically a wire antenna coil having multiple turns of electrically insulated single-strand or multi-strand platinum or gold wire. The electrical insulation of the internal coil 1236 can be provided by a flexible silicone molding. Various types of energy transfer, such as infrared (IR), electromagnetic, capacitive and inductive transfer, can be used to transfer the power and/or data from external device to cochlear implant. While the above description has described internal and external coils being formed from insulated wire, in many cases, the internal and/or external coils can be implemented via electrically conductive traces.

Example Implantable Stimulation System—Retinal Prosthesis

FIG. 13 illustrates a retinal prosthesis system 1301 that comprises, the first device 102, a retinal prosthesis 1300 and a mobile computing device 1303. The retinal prosthesis 1300 comprises a processing module 1325 (e.g., the second device 150) and a retinal prosthesis sensor-stimulator 1390 is positioned proximate the retina 1391 of a recipient. The first device 102 and the processing module 1325 can both include transmission coils 1156 aligned via respective magnet sets 110 and 152. Signals 1351 can be transmitted using the coils 1156.

In an example, sensory inputs (e.g., photons entering the eye) are absorbed by a microelectronic array of the sensor-stimulator 1390 that is hybridized to a glass piece 1392 including, for example, an embedded array of microwires. The glass can have a curved surface that conforms to the inner radius of the retina. The sensor-stimulator 1390 can include a microelectronic imaging device that can be made of thin silicon containing integrated circuitry that convert the incident photons to an electronic charge.

The processing module 1325 includes an image processor 1323 that is in signal communication with the sensor-stimulator 1390 via, for example, a lead 1388 which extends through surgical incision 1389 formed in the eye wall. In other examples, processing module 1325 can be in wireless communication with the sensor-stimulator 1390. The image processor 1323 processes the input into the sensor-stimulator 1390, and provides control signals back to the sensor-stimulator 1390 so the device can provide an output to the optic nerve. That said, in an alternate example, the processing is executed by a component proximate to, or integrated with, the sensor-stimulator 1390. The electric charge resulting from the conversion of the incident photons is converted to a proportional amount of electronic current which is input to a nearby retinal cell layer. The cells fire and a signal is sent to the optic nerve, thus inducing a sight perception.

The processing module 1325 can be implanted in the recipient and function by communicating with the first device 102 acting as an external device, such as a Behind-The-Ear (BTE) unit, a pair of eyeglasses, etc. The first device 102 can include an external light/image capture device (e.g., located in/on a BTE device or a pair of glasses, etc.), while, as noted above, in some examples, the sensor-stimulator 1390 captures light/images, which sensor-stimulator is implanted in the recipient.

Similar to the above examples, the retinal prosthesis system 1301 may be used in spatial regions that have at least one controllable network connected device associated therewith (e.g., located therein). As such, the processing module 1325 includes a performance monitoring engine 1327 that is configured to obtain data relating to a “sensory outcome” or “sensory performance” of the recipient of the retinal prosthesis 1300 in the spatial region. As used herein, a “sensory outcome” or “sensory performance” of the recipient of a sensory prosthesis, such as retinal prosthesis 1300, is an estimate or measure of how effectively stimulation signals delivered to the recipient represent sensor input captured from the ambient environment.

Data representing the performance of the retinal prosthesis 1300 in the spatial region is provided to the mobile computing device 1303 and analyzed by a network connected device assessment engine 1362 in view of the operational capabilities of the at least one controllable network connected device associated with the spatial region. For example, the network connected device assessment engine 1362 may determine one or more effects of the controllable network connected device on the sensory outcome of the recipient within the spatial region. The network connected device assessment engine 1362 is configured to determine one or more operational changes to the at least one controllable network connected device that are estimated to improve the sensory outcome of the recipient within the spatial region and, accordingly, initiate the one or more operational changes to the at least one controllable network connected device.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. An apparatus, comprising:
a first device having a first device magnet set of one or more magnets configured to provide a magnetic connection to secure the first device relative to a second device,
wherein the first device is configured to permit the first device magnet set to be selectively disposable by a user in the first device in first and second opposite orientations to change a strength of a magnetic securing force of the magnetic connection.

2. The apparatus of claim 1, further comprising:
a magnet case that defines a first volume and a second volume,
wherein the first device magnet set is disposed within the magnet case between the first volume and the second volume, and wherein the first volume is larger than the second volume.

3. The apparatus of claim 2, wherein the first device magnet set includes a first magnet and a second magnet, and wherein the magnet case further defines a third volume disposed between the first magnet and the second magnet.

4. The apparatus of claim 1, wherein the first device magnet set is disposed in a magnet case that defines a first half and a second half; and wherein the first half is more magnetic than the second half.

5. The apparatus of claim 1, wherein the one or more magnets are transversely magnetized.

6. The apparatus of claim 5, wherein the first device magnet set defines an axis perpendicular to a diameter, wherein the first device magnet set is configured to rotate about the axis.

7. The apparatus of claim 6, wherein the first device magnet set defines an axis perpendicular to a diameter, wherein the first device magnet set is configured to resist rotation about the axis.

8. The apparatus of claim 1, further comprising:
a magnet case defining a first end opposite a second end and housing the first device magnet set,
wherein while the magnet case is disposed in the first device in the first orientation, a first distance (d1) between the first end and a skin contact surface is greater than a second distance (d2) between the second end and the skin contact surface, and
wherein while the magnet case is disposed in the first device in the second orientation, the first distance (d1) is smaller than the second distance (d2).

9. The apparatus of claim 1, wherein the first device magnet set has a circular shape with a diameter parallel to a length of a skin contact surface of the apparatus.

10. The apparatus claim 1,
wherein the first device magnet set comprises a diametrically magnetized magnet.

11. A method, comprising:
establishing a first magnetic connection having a first strength between an implanted magnet set and an external magnet set while the external magnet set is disposed in a first orientation;
transitioning the external magnet set from being disposed in the first orientation to being disposed in a second orientation; and
establishing a second magnetic connection having a second strength between the implanted magnet and the external magnet set in the second orientation,
wherein the first strength is stronger than the second strength, and
wherein the transitioning includes rotating the external magnet set such that a different face of the external magnet set faces the implanted magnet when the second magnetic connection is established.

12. The method of claim 11, wherein the rotating includes rotating a magnet case that houses the external magnet set about an axis parallel to a diameter of the magnet case.

13. The method of claim 11, further comprising:
engaging in physical exercise; and
ceasing engaging in the physical exercise,
wherein the transitioning is responsive to ceasing engaging in the physical exercise.

14. The method of claim 11, wherein the transitioning includes:
removing the external magnet set from a magnet receptacle in the first orientation; and
replacing the external magnet set in the magnet receptacle in the second orientation.

15. The method of claim 11 wherein establishing the first magnetic connection includes wearing an external device in which the external magnet set is disposed; wherein the method further includes establishing an inductive power and data connection between the external device and an implanted device associated with the implanted magnet; or wherein rotating the external magnet set includes rotating the external magnet set 180 degrees.

16. A system comprising:
an external device comprising an external magnet set; and
an implantable device comprising an implantable magnet set;
wherein the external magnet set is configured to be selectively disposed in the external device in one of first and second different orientations to change a magnetic strength of a magnetic connection between the external magnet set and the implantable magnet set when the external device is disposed proximate the implantable device.

17. The system of claim 16, wherein the external magnet set comprises a first magnet and a second magnet, wherein the first magnet has a greater magnetic strength than the second magnet.

18. The system of claim 16, wherein one or more magnets of the external magnet set define a diameter; and wherein the one or more magnets are magnetized in a direction parallel to a plane of the diameter.

19. The system of claim 16, wherein the external magnet set or the implantable magnet set is configured to rotate in response to the magnetic connection.

20. The system of claim 16,
wherein one or more magnets of the external magnet set are axially magnetized.
